(19) United States
(12) Patent Application Publication
(10) Pub. No.: US 2012/0136293 A1
(43) Pub. Date: May 31, 2012

(54) SUPPORT FOR FOOT INJURIES

(76) Inventor: Mitunori Oosawa, Saga-shi (JP)

(21) Appl. No.: 13/261,132

(22) PCT Filed: Jul. 5, 2010
(86) PCT No.: PCT/JP2010/061732

§ 371 Date: Feb. 15, 2012

(30) Foreign Application Priority Data
Jul. 13, 2009 (JP) 2009-164864

(51) Int. Cl. A61F 5/00

(52) U.S. Cl. 602/28

(57) ABSTRACT

A support for overpronation is provided with a fitted support, which can be fitted on a foot, and a fastening belt. One end of the fastening belt is detachably fixed to a position in the fitted support that corresponds to the first metatarsophalangeal joint, the middle section of the belt is wound around the heel and the other end is detachably fixed to a position in the fitted support that corresponds to the Lisfranc joint and/or Chopart's joint. With the heel section as an anchor, external force is provided in opposing directions, between the first metatarsophalangeal joint and the Lisfranc joint, or the first metatarsophalangeal joint and Chopart's joint. The support for oversupination is fitted in the opposite way, providing external force in opposing directions.

### BACKGROUND OF THE INVENTION

The present invention relates to a supporter for foot disorders, suitable for improving foot disorders as represented by bunions.

There are generally known foot disorders such as bunions and plantar fasciitis, caused by foot motions under conditions such as being duck-footed or pigeon-toed, as well as from a foot with an excess or lack of pronating or supinating motions.

Traditionally, there have been proposed to rectify foot disorders such as bunions and plantar fasciitis, articles aiding weight shifting by an assistive device such as an insole (for example, in reference to Japanese Published Unexamined Patent Application No. 2001-104008), and articles that proactively apply an external force to joints of the foot (for example, in reference to Japanese Published Unexamined Patent Application No. 2000-308654 and Japanese Published Unexamined Patent Application No. 2001-262405).

However, in the case of Japanese Published Unexamined Patent Application No. 2001-104008, aging degradation (wear, deformation) of the insole easily occurs, and as it is for aiding weight shifting, rectifying effects limited to being supplemental do not contribute to fundamental improvement of foot disorders.

In the cases of Japanese Published Unexamined Patent Application No. 2000-308654 and Japanese Published Unexamined Patent Application No. 2001-262405, as the article does apply an inward pressing force on the first metatarsal joint and the fifth metatarsal joint, moderate effects can be expected. However, with only an inward pressing force, rectifying effects cannot always be considered sufficient.

In general, in the walking motion of a human, as shown in FIG. 13A, the heel lands on the ground (1), the sole contacts the ground (2), shifts to the first toe at the tip of the foot (3). With this walking weight shift mechanism that involves kicking the ground with the big toe, a walking line of a normal foot presents the trace shown in FIG. 13B. In FIG. 13B, first, the heel contacts the ground (1), and the body's center of gravity moves forward. While increasing weight-bearing, weight is supported by contacting the entire sole (2).

In this process, the movement involves a combination of a supinating motion and a plantar flexing motion, while the “outer vertical arch” mainly functions.

As the body's center of gravity moves forward further to the tip of the foot (3), the kicking process involves a combination of a pronating motion and a plantar flexing motion, while the “inner vertical arch” mainly functions.

Furthermore, when weight moves forward and the tip of the toes leave the ground (leave-ground), this is a plantar flexing motion, and the “side arch” mainly functions as weight is also applied and released through parts other than the big toe.

In contrast to the walking line of a normal foot mentioned above, the walking lines of overpronation and excessive supination, which are motions of an abnormal foot, are each shown in FIG. 13C and FIG. 13D.

With the walking line of overpronation in FIG. 13C, when compared to that of a foot with a normal arch as shown in FIG. 13B, it can be seen that weight is applied to the inner side. As shown in the same figure, in the weight support of a walker, there is no supinating motion, and pronating motions are vigorously performed, causing situations of increased excessive strain on the inner arch. By this excessive strain, during plantar flexing motions an inward pressure is applied to the sesamoid bone, causing abnormalities in the shape of the arch. One of the cases that accompany this abnormality in the shape of the arch, for example, is valgus of the big toe. Also in this case, motions of dorsal flexion are small, and floor contact does not start with the heel in many cases.

In this manner, when excessive strain on the inner arch increases, the plantar aponeurosis on the side becomes flaccid making each arch incapable of maintaining its height. In particular, the inner arch buckles, the talus is internally rotated, and the navicular bone, accompanied by inward projection, drifts into valgus, becoming the primary factor for various foot disorders.

Also, with the walking line of excessive supination in FIG. 13D, when compared to that of a foot with a normal arch as shown in FIG. 13B, it can be seen that weight is applied to the outer side. As shown in the same figure, in the weight support of a walker, when transferring from supinating motions to weight shifting motions, pronating motions become small. In the case of this excessive supination, an excessive strain is applied to the outer arch, leading to abnormalities in the shape of the arch. One of the cases that accompany this abnormality in the shape of the arch, for example, is varus of the little toe.

In this manner, when excessive strain on the outer arch increases, the arch of the outer side buckles. One of the cases occurring from this, for example, is the formation of a bunionette. This bunionette has a bad effect on the side arch as well, causing broadening of the space between metatarsal bones, which can eventually lead to an excessive supination type bunion. This excessive supination type is characterized by the excessive supination type being high in contrast to the shape of the instep being low. The inner arch is high, and the plantar arch is not flattened.

With the overpronation type and excessive supination type of the abnormal foot mentioned above, in contrast to the walking line of a normal foot, in the weight shift during walking, as shown in FIG. 13C and FIG. 13D, it can be seen that excessive stress is applied to the MP joint side of the first and fifth metatarsal bones.

With this excessive stress focusing on the metatarsal bones, depending on the insole described in Japanese Published Unexamined Patent Application No. 2001-104008 and the supporter described in Japanese Published Unexamined Patent Application No. 2000-308654 and Japanese Published Unexamined Patent Application No. 2001-262405 mentioned above, or a supporter, each weight shift could not be performed in the weight shifting while walking, thus causing a problem of preventing walking with the walking line of a normal foot.

In other words, in each of the inventions of Japanese Published Unexamined Patent Application No. 2001-104008 through Japanese Published Unexamined Patent Application No. 2001-262405 mentioned above, as each arch of the foot is shaped at rest, functions are limited to normalizing weight stress on the soles, and problems remain with their inability to properly act on walking weight shift while walking.

An object of the present invention is to provide a supporter for foot disorders capable of effectively rectifying foot disorders such as, for example, bunions caused by overpronation and excessive supination, as well as plantar fasciitis due to overpronation and excessive supination, with which it is possible to solve the problems of cases relating to abnormalities of the arch formed by a foot while walking.

### SUMMARY OF THE INVENTION

A supporter for foot disorders according to the present invention is provided with a fitting assisting device that can be worn on the foot, and a fastening belt in which one end is fixed onto or made detachable at a position corresponding to the first metatarsophalangeal joint on the fitting assisting device, the middle section wraps around the heel, and the other end is detachably fixed at a position corresponding to the Lisfranc joint and/or the Chopart joint on the fitting assisting device, wherein the supporter for foot disorders is made to apply an external force between the first metatarsophalangeal joint and the Lisfranc joint and/or the Chopart joint, anchored at the heel, in directions opposite to each other.

Also, in the supporter for foot disorders according to the present invention, as needed, one of the external forces in the opposing directions is a force in a direction that rotates the first metatarsophalangeal joint towards the inside of the foot, and the other is a force in a direction that rotates the Chopart joint towards the outside of the foot.

Also, in the supporter for foot disorders according to the present invention, as needed, one of the external forces in the opposing directions is a force in a direction that rotates the first metatarsophalangeal joint towards the outside of the foot, and the other is a force in a direction that rotates the Chopart joint towards the inside of the foot.

Also, in the supporter for foot disorders according to the present invention, as needed, the fastening belt is fixated at the heel where the middle section is wrapped around, and each of the backsides between the fixated middle section and one end, as well as between the middle section and the other end, is shaped as a smooth surface against the surface of the fitting assisting device.

According to the present invention, it is possible to provide a supporter for foot disorders capable of effectively rectifying foot disorders such as, for example, bunions caused by overpronation and excessive supination resulting from abnormalities in the arch, as well as plantar fasciitis due to overpronation and excessive supination. This supporter for foot disorders is easy to handle, and normalizes arches of the foot shaped while walking by improving the way that weight is applied to the foot, which in turn is done by normalization of pronating and supinating foot motions while walking. Additionally, by rectifying and improving overpronation and excessive supination in normal walking motions, various physical health conditions caused by disorders in walking motions can be improved, thereby advancing one's health and promoting beauty as well.

### BRIEF DESCRIPTION OF THE DRAWINGS

FIG. 1 is an external perspective view of the supporter for foot disorders.

FIG. 2 is an external perspective view showing the supporter for foot disorders fitted on the right foot for rectifying overpronation.

FIG. 3 is a top view showing the supporter for foot disorders fitted on the right foot for rectifying overpronation.

FIG. 4 is a bottom view showing the supporter for foot disorders fitted on the right foot for rectifying overpronation.

FIG. 5 is a side view showing the supporter for foot disorders fitted on the right foot for rectifying overpronation.

FIG. 6 is a side view showing the supporter for foot disorders fitted on the left foot for rectifying overpronation.

FIG. 7 is an external view, from the heel side, of the supporter for foot disorders fitted on the right foot for rectifying overpronation.

FIG. 8 is an explanatory drawing of the actions in rectifying overpronation.

FIG. 9 is an external perspective view showing the supporter for foot disorders fitted on the right foot for rectifying excessive supination.

FIG. 10 is a top view showing the supporter for foot disorders fitted on the right foot for rectifying excessive supination.

FIG. 11 is a bottom view showing the supporter for foot disorders fitted on the right foot for rectifying excessive supination.

FIG. 12 is an explanatory drawing of the actions of the supporter for foot disorders fitted on the right foot for rectifying excessive supination.

FIG. 13A, FIG. 13B, FIG. 13C and FIG. 13D are explanatory drawings showing the walking motion and walking mechanism of a human.

### DETAILED DESCRIPTION OF THE INVENTION

Hereinafter, an embodiment of the present invention will be explained.

### (Configuration)

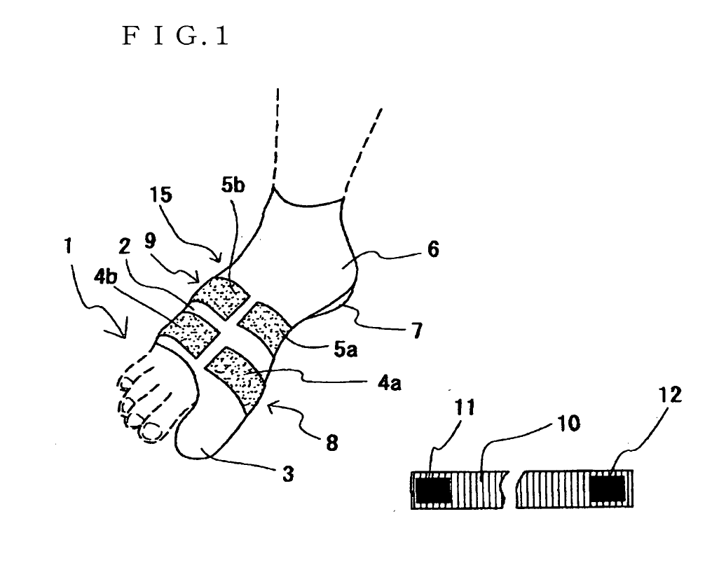

As shown in FIG. 1, a supporter for foot disorders 100 can be roughly divided into a fitting assisting device 2 and a fastening belt 10.

The fitting assisting device 2 is of a material having moderate flexibility such as cloth, and is cylindrical overall to cover a foot 1. On the front end of the fitting assisting device 2, a big toe insertion part 3 is provided, and on the rear end, for hooking on to the heel 7, a heel wrapping part 6 is provided. The big toe insertion part 3 prevents the fitting assisting device 2 from slipping towards the heel 7 when wearing the fitting assisting device 2. On the other hand, the wrapping part 6 acts in preventing the fitting assisting device 2 from slipping towards the toes.

The shape of the fitting assisting device 2 is not limited to the above mode, and may be of a sock-like pouch shape. For the material of the fitting assisting device 2, a material of low elasticity is preferable. That is because, as will be described later, strength is required to resist the external force for rectification being applied to the fitting assisting device 2 by the fastening belt 10.

The fitting assisting device 2 is provided with belt catches 4a, 4b, 5a and 5b. The belt catches 4a and 4b are configured in a position corresponding to the first metatarsophalangeal joint (or the head of the fifth metatarsal bone) 8 of the foot 1 when the fitting assisting device 2 is fitted on the foot 1. The belt catches 5a and 5b are arranged in a position corresponding to the Lisfranc joint (or the base of the fifth metatarsal bone) 9 and/or the Chopart joint 15 when the fitting assisting device 2 is worn.

The fastening belt 10 is of a material having stretchability (elasticity) such as a cloth belt containing rubber or a rubber belt. Both ends of the fastening belt 10 are arranged with fixation parts 11 and 12 that can be detachably fixed in a removable manner to the belt catches 4a, 4b, 5a, and 5b of the fitting assisting device 2. The fixation parts 11 and 12 are to be paired with the belt catches 4a, 4b, 5a, and 5b of the fitting assisting device 2 to fix the fastening belt 10 in place, and for example, materials such as Velcro (registered trademark) may be used.

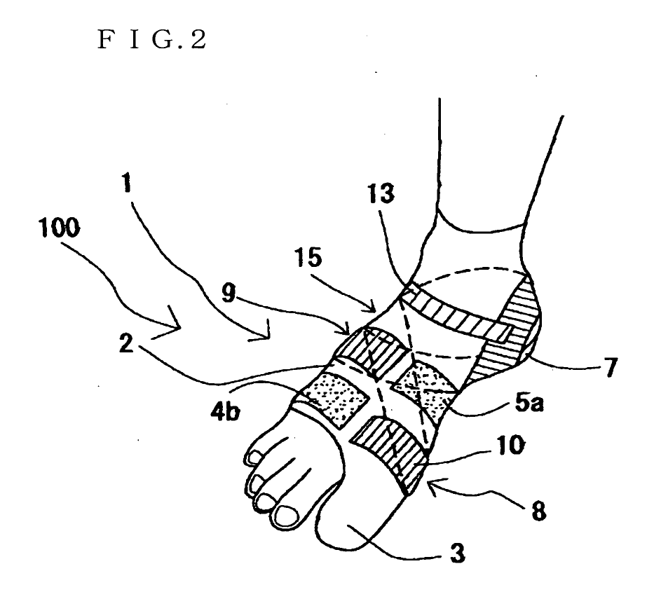

Other than the above, as shown in FIG. 2, to prevent the fastening belt 10 from being undone in the heel 7 when the fastening belt 10 is worn, use of a slippage prevention belt 13 that can be wrapped around the ankle is preferable.

### (Fitting Method in Rectifying Overpronation)

With the above structure, a fitting method of the supporter for foot disorders 100, in a case of rectifying overpronation as an example of a case caused by abnormalities of the arch of the foot shaped while walking, will be explained below.

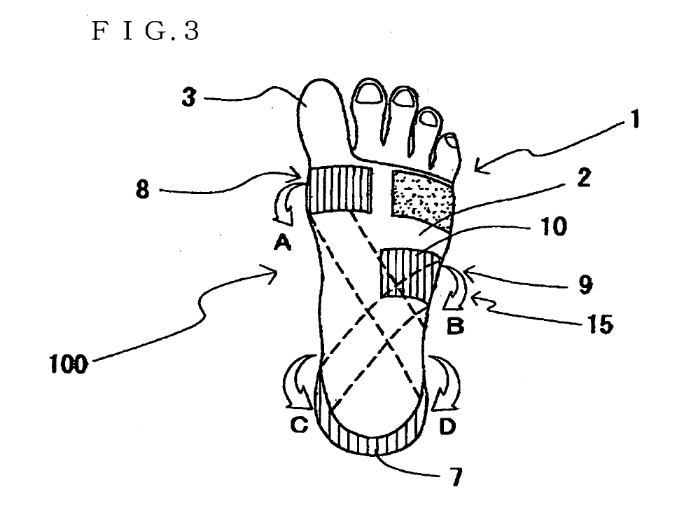

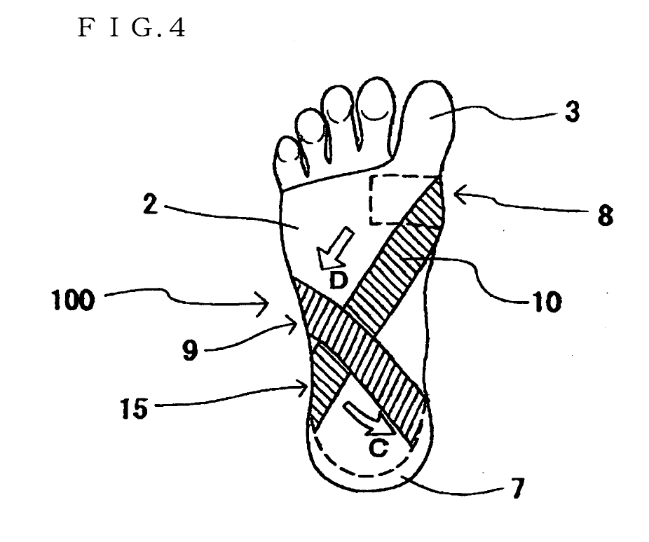

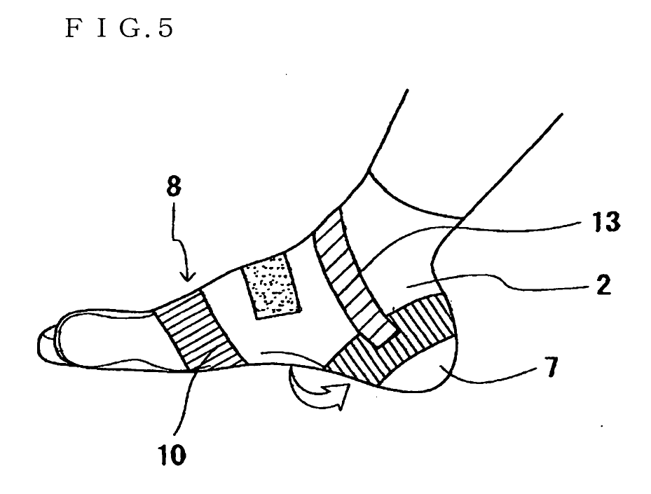

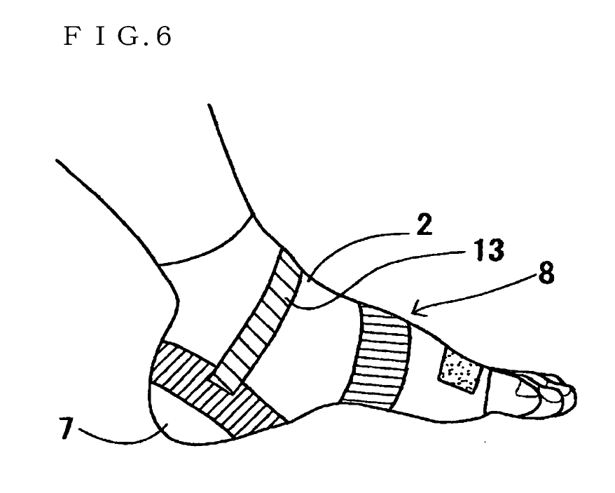

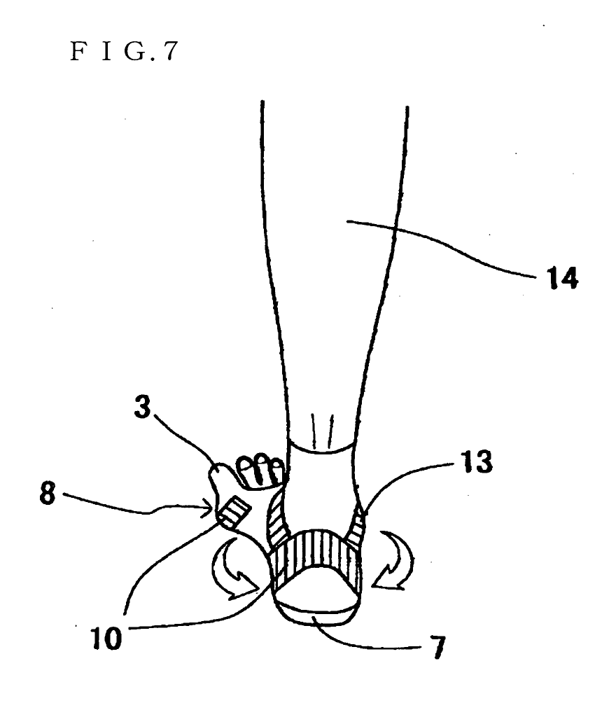

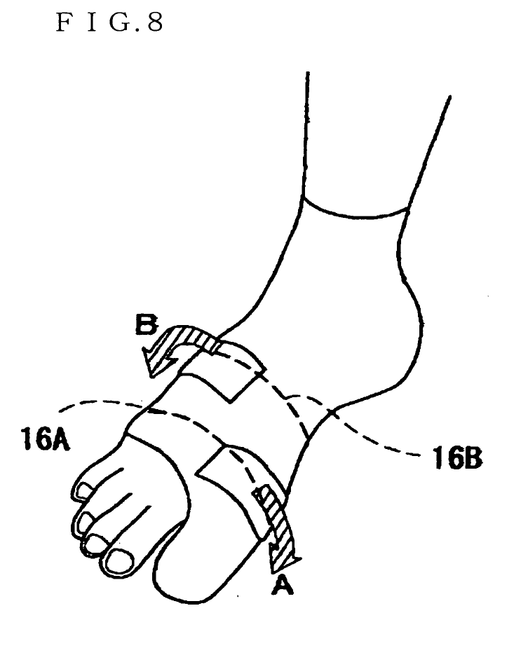

FIG. 1 is an external perspective view of the supporter for foot disorders, FIG. 2 is an external perspective view showing the supporter for foot disorders fitted on the right foot, FIG. 3 is a top view showing the supporter for foot disorders fitted on the right foot, FIG. 4 is a bottom view showing the supporter for foot disorders fitted on the right foot, FIG. 5 is a side view showing the supporter for foot disorders fitted on the right foot, FIG. 6 is a side view showing the supporter for foot disorders fitted on the left foot, FIG. 7 is an external view, from the heel side, of the supporter for foot disorders fitted on the right foot, and FIG. 8 is an explanatory drawing of the actions.

FIG. 3 shows the view from the top of the right foot. Upon fitting the supporter for foot disorders 100, first, the fitting assisting device 2 is worn on the foot 1. In doing so, the big toe insertion part 3 is placed to cover the big toe while the wrapping part 6 is wrapped around the heel 7 to fix it in place (FIG. 1, FIG. 2, and FIG. 3).

Next, the fixation part 11 of the fastening belt 10 is fixed onto the belt catch 4a, and while stretching out the fastening belt 10, is wrapped around the heel 7 (FIGS. 3, 5, and 7) via the sole of the foot 1 in the direction going from the outer side (FIG. 3, right side) to the inner side (FIG. 3, left side) of the foot 1. From there, the fastening belt 10 is taken around the sole of the foot 1 again before being pulled towards the outer side of the foot 1 where the fixation part 12 is fixed onto the belt catch 5b. Then, the slippage prevention belt 13 is worn around the ankle to fix the position of the fastening belt 10, wrapped around the heel 7, onto the side of the heel 7 (FIGS. 2 and 5).

In such way, the supporter for foot disorders 100 can be worn in an extremely simple manner by first wearing the fitting assisting device 2 on the foot 1, followed by wrapping the fastening belt 10.

The above is an example for the right foot, and the same applies for the left foot. This can be thought of in a symmetrical manner, and with the outline shown in FIG. 6, further explanation of the details will be omitted.

### (Operation)

Next, the Effects that the Fitted Supporter for foot disorders 100 has on the foot 1 will be explained.

As shown in FIG. 3, a tractive force is exerted on the fastening belt 10 stretched out upon wearing. By this tractive force, anchored at the section where the fastening belt 10 is wrapped around the heel 7, an external force is applied in opposite directions between the position of the belt catch 4a (in other words, the first metatarsophalangeal joint 8) and the position of the belt catch 5b (in other words, the Lisfranc joint 9 and/or the Chopart joint 15).

Specifically, to the first metatarsophalangeal joint 8, between the fixation part 11 of the fastening belt 10 fixed onto the belt catch 4a and the section where the belt is wrapped around the heel 7, a tractive force in the direction of arrow D that is anchored at the heel 7 works in applying an external force on the first metatarsophalangeal joint 8 twisting it towards the inner side of the sole of the foot 1 (refer to arrow A in the figure). The external force applied in this manner acts in opening the head (the front part) of the first metatarsal bone towards the inner side of the foot 1.

At the same time, to the Lisfranc joint 9 and/or the Chopart joint 15, between the fixation part 12 of the fastening belt 10 fixed onto the belt catch 5b and the section where the belt is wrapped around the heel 7, a tractive force in the direction of arrow C that is anchored at the heel 7 works in applying an external force on the Lisfranc joint 9 and/or the Chopart joint 15 twisting it towards the sole of the foot 1 in the outer side (refer to arrow B in the figure).

The external force applied in this manner acts in pressuring the base of the fifth metatarsal bone towards the inner side of the foot 1. Also, similarly, this external force acts in pressuring the area around the cuboid bone of the Chopart joint 15 towards the inner side of the foot 1.

By acting on the foot 1 as indicated by the arrow A and the arrow B, as shown in FIG. 8, the side arch 16A and the tarsal bone arch 16B of the foot 1 can be shaped, improving as well as preventing various foot disorders caused by overpronation while walking.

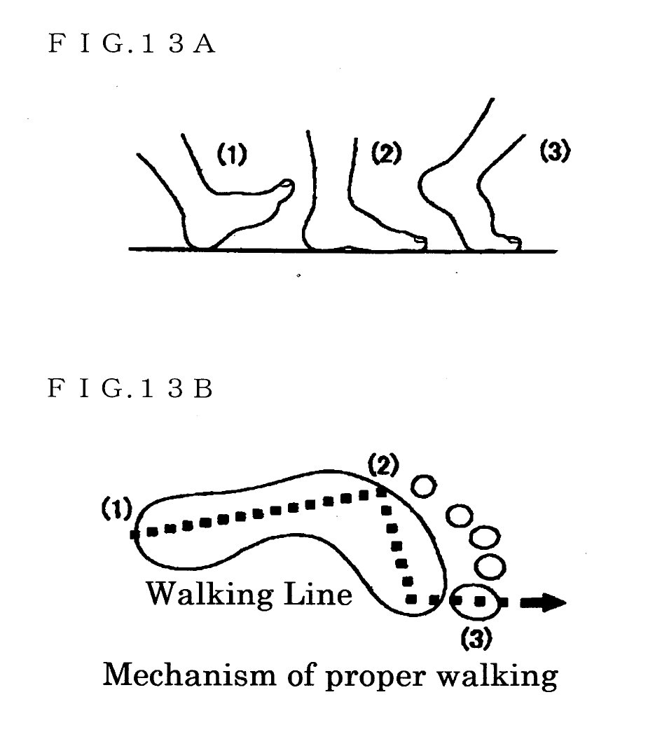

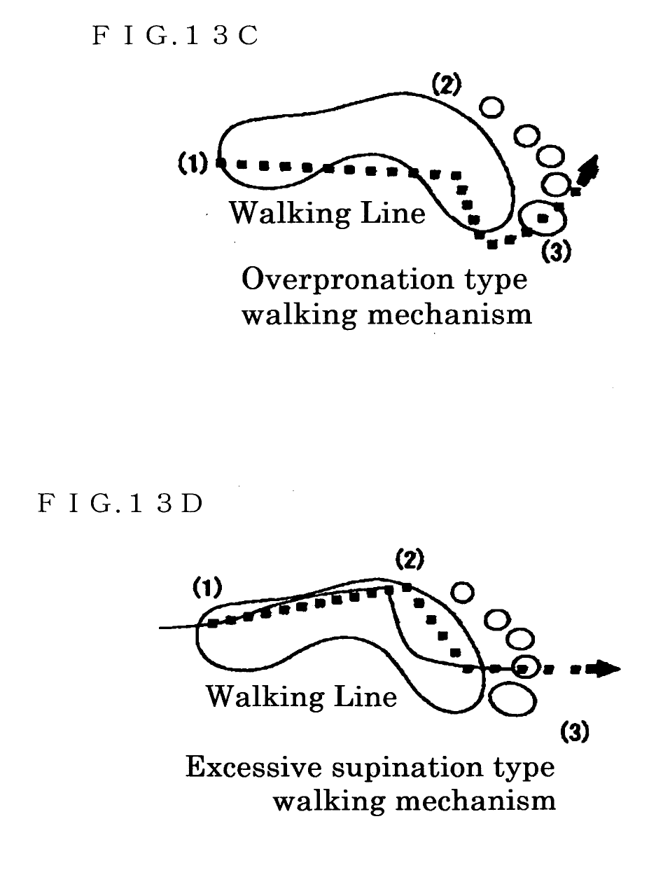

As stated above, with the supporter for foot disorders 100, while wearing the supporter for foot disorders 100, an external force can be constantly applied in opposite directions (in this example, in directions away from one another) between the first metatarsophalangeal joint 8 and the Lisfranc joint 9 and/or the Chopart joint 15. As a result, unlike an insole that only acts intermittently while walking when weight is applied, an external force is actively applied continuously in the entire sequence of motions in walking from the landing of the heel (refer to (1) of FIG. 13A) to the kicking by the toe (refer to (3) of FIG. 13A), allowing for effective rectification of overpronation.

### (Usage Method in Rectifying Excessive Supination)

Next, the fitting method of the supporter for foot disorders 100, in a case of rectifying excessive supination related to abnormalities of the arch of the foot shaped while walking, will be explained.

When rectifying excessive supination, the fastening belt 10 is fitted to apply an external force in a direction completely opposite of when rectifying overpronation.

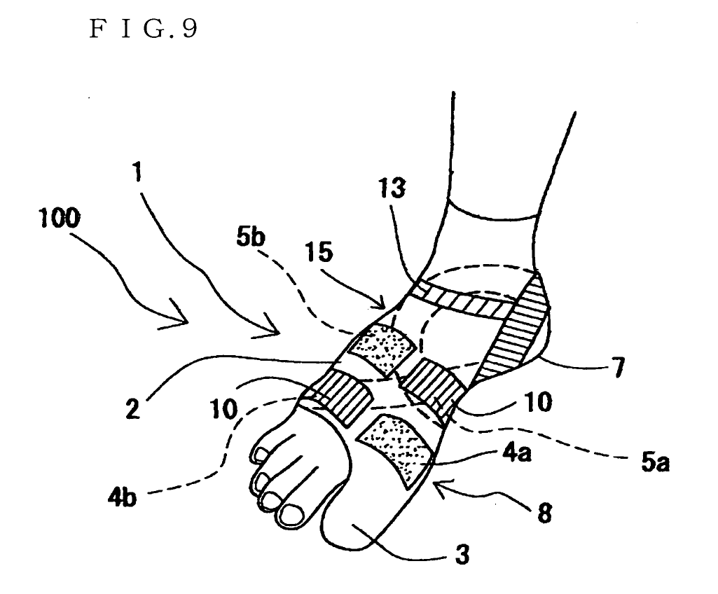

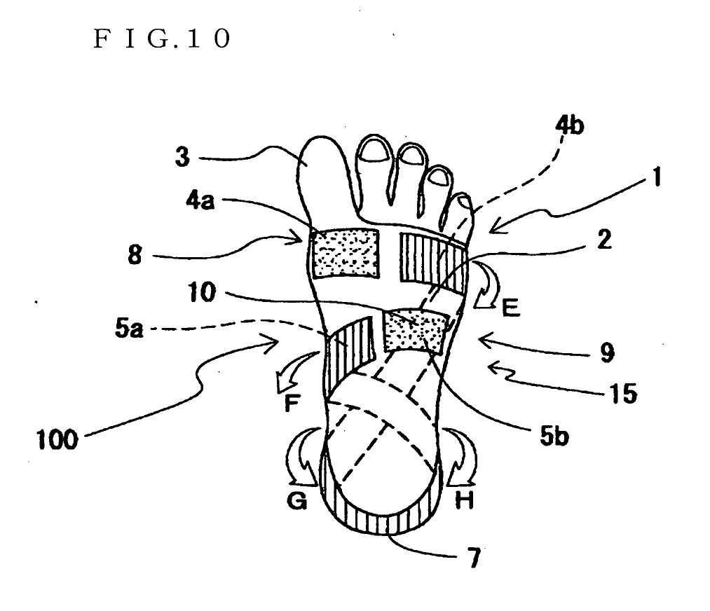

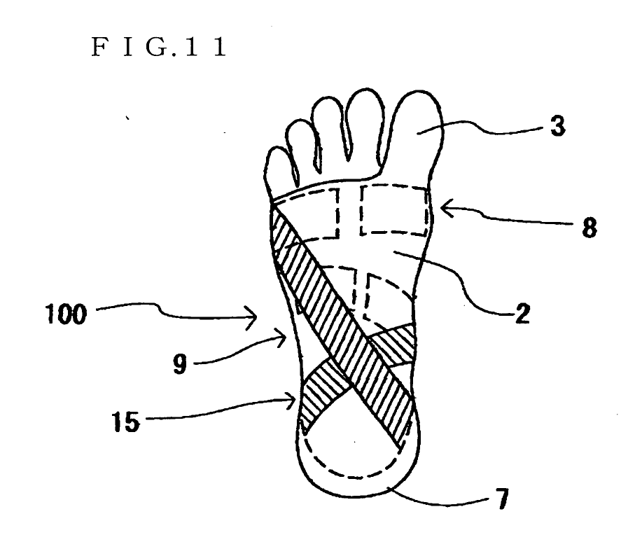

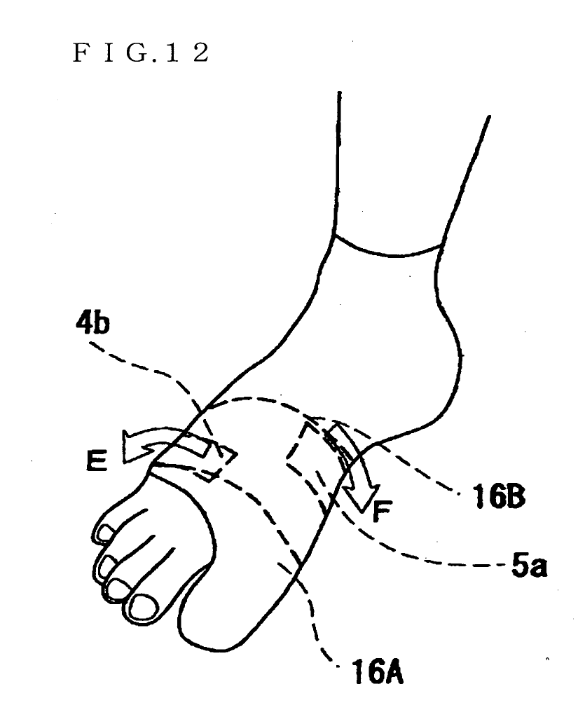

FIG. 9 is an external perspective view of the supporter for foot disorders 100, FIG. 10 is a top view of foot 1, FIG. 11 is a bottom view of foot 1, FIG. 12 is an explanatory drawing of the actions of the supporter for foot disorders 100, and FIG. 13 shows an explanatory drawing showing the walking motion and walking mechanism of a human.

Upon wearing the supporter for foot disorders 100, similar to when rectifying overpronation, first, the fitting assisting device 2 is worn on the foot 1, then the big toe insertion part 3 is placed to cover the big toe while the wrapping part 6 is wrapped around the heel 7 to fix it in place (FIG. 1, FIG. 9, and FIG. 10).

Next, the fixation part 11 of the fastening belt 10 is fixed onto the belt catch 4b (FIG. 11), and while stretching out the fastening belt 10, it is stretched along the sole of the foot 1 from the outer side (FIG. 10, right side) to the inner side (FIG. 10, left side), wrapped clockwise around the heel 7, then stretched towards the inner side of the foot 1 via the backside of the foot 1, where the fixation part 12 is fixed onto the belt catch 5a (FIG. 10 and FIG. 11). Although the above is an example for the right foot, the same applies for the left foot and can be thought of in a symmetrical manner.

### (Effect)

Next, the effects that the fitted supporter for foot disorders 100 has on the foot 1 will be explained.

As shown in FIG. 10, a tractive force is exerted on the fastening belt 10 stretched out upon wearing. By this tractive force, to the fifth metatarsophalangeal joint, between the fixation part 11 of the fastening belt 10 fixed onto the belt catch 4b and the section where the belt is wrapped around the heel 7, a tractive force in the direction of arrow G that is anchored at the heel 7 works in applying an external force on the fifth metatarsophalangeal joint twisting it towards the outer side of the sole of the foot 1 (refer to arrow E in the figure). The external force applied in this manner acts in opening the head (the front part) of the fifth metatarsal bone towards the outer side of the foot 1.

At the same time, to the Lisfranc joint 9 and/or the Chopart joint 15, between the fixation part 12 of the fastening belt 10 fixed onto the belt catch 5a and the section where the belt is wrapped around the heel 7, a tractive force in the direction of arrow H that is anchored at the heel 7 works in applying an external force on the Lisfranc joint 9 and/or the Chopart joint 15, twisting it counterclockwise in a downwards direction in the inner side of the foot 1 (refer to arrow F in the figure).

The external force applied in this manner acts in pressuring the base of the fifth metatarsal bone downwards towards the outer side of the foot 1. Also, similarly, this external force acts in pressuring the navicular bone and the area around the cuneiform bone of the Chopart joint 15 in a downwards direction of the foot 1.

In this manner, by wrapping the fastening belt 10, the fitting state as shown in FIG. 9 is created, and external forces act in opposite directions in such way that the fifth metatarsophalangeal joint is twisted clockwise and pulled downwards, while the Lisfranc joint 9 and/or the Chopart joint 15 is twisted counterclockwise and pulled downwards.

By acting on the foot 1 as indicated by the arrow E and arrow F, as shown in FIG. 12, the side arch 16A and the tarsal bone arch 16B of the foot 1 can be shaped, improving as well as preventing various foot disorders caused by excessive supination while walking.

As stated above, with the supporter for foot disorders 100, while wearing the supporter for foot disorders 100, an external force can be constantly applied in opposite directions (in this example, in directions each opposite of when rectifying overpronation as described above) between the fifth metatarsophalangeal joint and the Lisfranc joint 9 and/or the Chopart joint 15. As a result, unlike an insole that only acts intermittently while walking when weight is applied, an external force is actively applied continuously in the entire sequence of motions in walking from the landing of the heel (refer to (1) of FIG. 13A) to the kicking by the toe (refer to (3) of FIG. 13A), allowing for effective rectification of excessive supination.

### (Variations of the Fastening Belt)

Furthermore, the fastening belt 10 described above, may use belts of various materials and structures generally known as a belt. Although not limited, the fastening belt 10 is fixated at the heel 7 where the middle section is wrapped around, and each of the backsides between the fixated middle section and one end, as well as between the middle section and the other end, may be shaped as a smooth surface against the surface of the fitting assisting device 2. In this manner, by the fastening belt 10 being shaped to be a smooth surface against the surface of the fitting assisting device 2, frictional resistance generated by contact of the fastening belt 10 with the fitting assisting device 2 is reduced, and wearing comfort and ease of movement are improved. This allows for walking with less feeling of fatigue, even when walking for longer periods of time while wearing the supporter for foot disorders.

Furthermore, although in each of the above embodiments, the fastening belt 10 is configured to be a single band-shaped belt, this may also be configured as double-band-shaped belts starting from the heel 7 of the wearer. In this case, one end of each of the two fastening belts 10 are either sewn on to the heel 7, or attached in a removable manner by such as a hook and loop fastener.

Furthermore, in each of the above embodiments, by rectifying and improving overpronation and excessive supination in normal walking motions, various physical health conditions caused by disorders in walking motions can be improved, one's health is advanced, as well as promoting beauty by shaping the waistline and hips through improvement of the hips having a close connections with the feet.

Furthermore, in each of the above embodiments, by rectifying and improving overpronation and excessive supination in normal walking motions, excess motions can be reduced and the foot can be motioned efficiently, thus improving the capability to use the foot. The center of gravity in the foot is stabilized, and running ability is also improved while being stabilized, thus improving the athletic ability of athletes that use their feet in sports such as baseball, golf, and soccer.

Furthermore, in each of the above embodiments, although a fitting assisting device and a fastening belt were used individually as the supporter for foot disorders, this invention is not limited to this embodiment. For example, by using a pile-like fabric sewn on or a hook and loop fastener, a form such as a sock, stocking, or slipper having a structure where the fitting assisting device is woven so as to be integrated with the belt catches, can also be considered as an embodiment.

In such embodiments, regarding the external forces in rectifying and improving overpronation and excessive supination in normal walking motions mentioned above, when compared to the embodiments listed above, strengths are slightly inferior. On the other hand, ease of wear and removal is improved. This ease of wear and ease in handling are especially effective in cases where the supporter needs to often be worn and removed.

1. A supporter for foot disorders, comprising: a fitting assisting device that can be worn on the foot; and a fastening belt in which one end is fixed onto or made detachable at a position corresponding to the first metatarsophalangeal joint on the fitting assisting device, the middle section wraps around the heel, and the other end is detachably fixed at a position corresponding to the Lisfranc joint and/or the Chopart joint on the fitting assisting device, wherein
the supporter for foot disorders is made to apply an external force twisting between the first metatarsophalangeal joint and the Lisfranc joint and/or the Chopart joint, anchored at the heel, in directions opposite to each other, and
one of the said external forces twisting in the opposing directions is a force in a direction that rotates the first metatarsophalangeal joint towards the inside of the foot, and the other is a force in a direction that rotates the Lisfranc joint and/or the Chopart joint towards the outside of the foot.

2.-3. (canceled)

4. The supporter for foot disorders according to claim 1, wherein
the fastening belt is fixated at the heel where the middle section is wrapped around, and each of the backsides between the fixated middle section and one end, as well as between the middle section and the other end, is shaped as a smooth surface against the surface of the fitting assisting device.

5. A supporter for foot disorders, comprising: a fitting assisting device that can be worn on the foot; and a fastening belt in which one end is fixed onto or made detachable at a position corresponding to the fifth metatarsophalangeal joint on the fitting assisting device, the middle section wraps around the heel, and the other end is detachably fixed at a position corresponding to the Lisfranc joint and/or the Chopart joint on the fitting assisting device, wherein
the supporter for foot disorders is made to apply an external force twisting between the fifth metatarsophalangeal joint and the Lisfranc joint and/or the Chopart joint, anchored at the heel, in directions opposite to each other, and
one of the external forces twisting in the opposing directions is a force in a direction that rotates the fifth metatarsophalangeal joint towards the outside of the foot, and the other is a force in a direction that rotates the Lisfranc joint and/or the Chopart joint towards the inside of the foot.

6. The supporter for foot disorders according to claim 5, wherein the fastening belt is fixated at the heel where the middle section is wrapped around, and each of the backsides between the fixated middle section and one end, as well as between the middle section and the other end, is shaped as a smooth surface against the surface of the fitting assisting device.

7. The supporter for foot disorders according to claim 1, wherein the fastening belt is formed of an elastic member.

8. The supporter for foot disorders according to claim 5, wherein the fastening belt is formed of an elastic member.
